(19) United States
(12) Patent Application Publication
(10) Pub. No.: US 2024/0216757 A1
(43) Pub. Date: Jul. 4, 2024

(54) SYSTEM AND METHOD FOR BIOLOGICAL FEEDBACK MEASUREMENT FROM VIDEO

(71) Applicant: Vilnius Gediminas Technical University, Vilnius (LT)

(72) Inventors: Arturas SERACKIS, Vilnius (LT); Kristina DAUNORAVICIENE, Vilnius (LT); Andzela SESOK, Vilniaus raj (LT); Rytis MASKELIUNAS, Kauno raj (LT); Darius PLONIS, Vilnius (LT); Julius GRISKEVICIUS, Vilnius (LT)

(21) Appl. No.: 18/148,554

(22) Filed: Dec. 30, 2022

(51) Int. Cl. A63B 24/00; G06T 7/70; G06T 17/00; G06V 10/74; G06V 10/764; G16H 50/20

(52) U.S. Cl. CPC A63B 24/0003; G06T 7/70; G06T 17/00; G06V 10/761; G06V 10/764; G16H 50/20; A63B 2024/0009; G06T 2207/20044

(57) ABSTRACT

The present embodiment provides a biological feedback measurement system and method for capturing, analyzing, and presenting users (11) exercise data. Comprises video cameras (10a, 10b, 10c) surrounding the user (11), capturing the user's (11) movements, processing, and predicting 3D coordinates of human skeleton joints. Comprise a synchronization system and automatic calibration algorithm uses a distance metric to estimate the distance between joints. A shared hub (12) with a camera automatic calibration algorithm is provided to map multiple human skeletons into a single common world coordinate system, processing, identifying, and correcting joint position prediction failures from a sequence of skeleton joint positions detected in consecutive video frames and a vector of features from the changes in coordinate values for each joint. The system uses a machine learning classifier to identify the type of exercise and a fuzzy logic-based system to map subjective evaluations to the parameters measured, displays medical professional evaluations, provides feedback to the user (11).

### FIELD OF THE INVENTION

This invention involves a system and method for recognizing, analyzing, and displaying the movement and positional information concerning the joins of the human skeleton from video and applying mathematical models to provide biological feedback, valuable to a patient (user).

### BACKGROUND OF THE INVENTION

People often need to perform various physical exercises and movements to improve their physical and cognitive health. Techniques for measuring and quantifying human pose and movement dynamics are known. Traditional techniques for measuring human pose and movement dynamics rely on markers or sensors attached to the patient's body or clothing (US2016089075, U.S. Pat. No. 9,311,789B1, US2019336066). These approaches are intrusive and uncomfortable for the patient, as well as expensive and time-consuming for the therapist.

One of close prior art to present invention is US2013123667 (A1) 2013 May 16. There movements are measured, an avatar is used to provide an example of movement. This system measures, shows the result, collects data, but there is no detailed decision-making mechanism, exercise comparison or evaluation mechanism.

One more similar system to presented invention US2019091515 (A1) 2019 Mar. 28. For monitoring the performance of exercises, to connect with the exercise stored in the database, to provide feedback on the accuracy of the exercise performance, there is used a depth camera, a specialized sensing camera. The feedback in this patent focuses on giving advice on how to perform the exercise better. This patent has an exercise recognition stage. In this patent, recognition is carried out by comparing the initial positions of the body (the position of the joints at the beginning of the exercise).

There is no artificial intelligence to predict the depth from the image. In this case, biological feedback is provided, which describes the state of a person, its change from previous measurements. In addition, the solution can provide/show how much the performed movement differs from the desired—goal in a specific exercise. But there is no recognition, a machine learning (artificial intelligence) module, by which the input gives the variation of joint coordinates in several consecutive video frames.

Also this application claims benefit of US2020242805 (A1) 2020 Jul. 30, CALIBRATING CAMERAS USING HUMAN SKELETON. Where a camera system comprising, multiple cameras may be used to observe an area from different perspectives. Deficiency of this invention are: that there is used camera with depth sensor to detect a person also determination of the plane (in its world coordinate system) on which a person is standing, attempts the geometrically calculation the positions of the points of the skeleton—similar to the theory of multiple view or stereo view geometry, it need to be solved problem of separating a specific person from others also looks for additional characteristic points (features) that have images for overlapping (similar to photogrammetry).

Analogue has several redundant elements, without which the necessary positioning of the cameras is achieved in presented invention.

The difference from previous inventions is that present invention involves predicting depth from each camera view, with some points having a lower uncertainty and others having a higher uncertainty (probability of error and bounds on the variation of errors/deviations). An iterative algorithm such as Least mean squares is utilized to combine the coordinates (position) of the center of the coordinates of the remaining cameras to the human skeleton points in the coordinate system of one selected camera so that the x, y, z coordinates of the selected skeleton points gets as close as possible. An objective function is chosen, where according to one of the possible distance metrics (e.g., Euclidean), the distance between the skeleton points obtained from different cameras is minimized. Targeting is provided only when one person enters the field of view of the cameras, with no need to find additional characteristic points (features) used to align images (like photogrammetry). The alignment instead is done solely by using human skeleton joints (points).

### SUMMARY OF THE INVENTION

The following embodiments and aspects thereof are described and illustrated in conjunction with system, tools and method which are meant to be exemplary and illustrative, not limiting in scope.

There is provided biological feedback measurement system for capturing, analyzing, and presenting users exercise data comprising: at least two video cameras surrounding the user, capturing the user's movements and processing and predicting 3D coordinates of up to 32 connected human skeleton joints; a shared hub with a camera automatic calibration algorithm to map multiple human skeletons from different origin points into a single common world coordinate system, processing, identifying and correcting joint position prediction failures from a sequence of skeleton joint positions detected in consecutive video frames and a vector of features from the changes in coordinate values for each joint; a machine learning classifier to identify the type of exercise; a fuzzy logic-based system to map subjective evaluations to the parameters measured; a graphical user interface displaying the monitored joints on a 3D mannequin model; medical professional evaluations.

Hub further comprises a synchronization system.

The camera automatic calibration algorithm uses a distance metric to estimate the distance between joints.

The camera automatic calibration algorithm uses human skeleton joints of the same type to minimize a pre-defined cost function for joint positioning.

The camera automatic calibration algorithm further comprises an iterative process and a cost function.

The graphical user interface displays a range to indicate the progress and distance to desired values.

A method of biological feedback measurement for calibrating and monitoring the users execution of an exercise, comprising: capturing the user's movements with a plurality of video cameras around a user performing an exercise; using an image processing process to predict the 3D coordinates of connected human skeleton joints; sending the predicted joint coordinates, along with additional camera synchronization information, to a shared hub; using a camera automatic calibration algorithm to map human skeletons from different origin points into a single common world coordinate system; identifying and correcting joint position prediction failures; taking a sequence of skeleton joint positions detected in consecutive video frames and computing a vector of features from the changes in coordinate values for each joint; analyzing the features of the joints from all cameras to identify the joints with the lowest levels of dynamical changes in coordinates; selecting joints of the same type (position in the skeleton) for camera automatic calibration; estimating the camera position in the world coordinate system; using a cost function to minimize the distance between the joint group; assigning positions of the remaining joints using the camera frames that best capture the motions of those joints; providing the human motion analysis and parameter extraction block with all indicated human skeleton joints in the right, fused positions; calculating parameters that a medical specialist has identified and must be followed during the exercise; using a machine learning classifier to identify the type of exercise the patient is working on; using numerical data such as movement of the skeleton joints, changes in the angle between joints during a single exercise phase, motion plane angle relative to the human body plane, motion magnitude and other associated features as input features to the classifier; selecting a set of parameters to be monitored during the exercise; using a fuzzy logic-based system to map subjective evaluations to the parameters that is measured; displaying visual simulations of medical professional evaluations and comparing current joint motion, angle changes and other parameters of past values related to the patient; and displaying the monitored joints on a 3D mannequin model that follows the patient's movements, as well as measured angles.

The step of selecting a set of parameters to be monitored during the exercise is pre-determined by a medical professional before the exercise.

The decision rules connecting the inputs to the outputs is pre-defined for every exercise type.

The step of using a machine learning classifier to identify the type of exercise the patient is working on is conducted on a list of exercises recommended by a medical professional for a specific patient.

The step of using a fuzzy logic-based system to map subjective evaluations to the parameters that is measured includes calculating the membership function parameters automatically by fitting the data to historical measurements and subjective evaluations.

The step of displaying visual simulations of medical professional evaluations includes presenting the evaluations in the form of graphs, color bars, numbers, and binary indicators.

### BRIEF DESCRIPTION OF THE DRAWINGS

The provided system and method will be better understood from the following detailed descriptions together with detailed accompanying drawings, wherein:

FIG. 1 shows an example of an application of a preferred embodiment.

FIG. 2 shows a schematic representation of camera autocalibration.

FIG. 3 shows a schematic representation of the components of the system of the present invention.

FIG. 4 shows a schematic representation of exercise type recognition.

### DETAILED DESCRIPTION

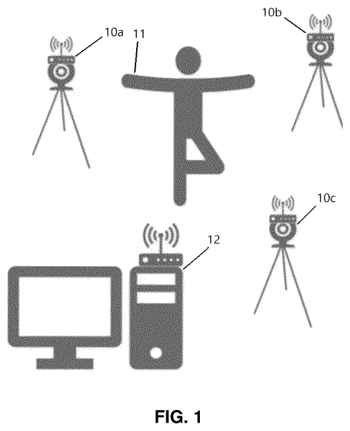

An example reference to FIG. 1 an application of a preferred embodiment of the invention, depicting a user 11 in a room. The user 11 is surrounded by video cameras devices 10a, 10b, 10c and performs a group of exercises recommended by the medical professional. Each video camera 10a, 10b, 10c runs an image processing process where a machine learning model is used to predict 3D coordinates for each of 17 connected human skeleton joints. The predicted joint coordinates are saved in coordinate system with origin point position, individually for each camera 10a, 10b, 10c. The coordinates together with additional information for camera synchronization from each camera 10a, 10b, 10c are transmitted into a common hub 12 (computer device) where a special iterative camera automatic calibration algorithm is applied. The camera automatic calibration algorithm maps three human skeletons, obtained and saved in different coordinate systems (origin points) into one common world coordinate system. Joint position prediction failures are identified by a process introduced in this invention and the required corrections are made. The automatic calibration process takes a sequence of skeleton joint positions detected in consecutive video frames, calculates a vector of features from coordinate value changes for each joint. As a feature set, the features that represents joint movement dynamics (such as variance, standard deviation, difference between maximum and minimum value, etc.) are selected. A fixed number of joints with lower level of dynamical changes of the coordinates are selected by analyzing features of joints, collected from all cameras 10a, 10b, 10c. Joints of the same type (position in the skeleton) are selected for camera automatic calibration—estimation of camera position in world coordinate system. During the iterative process, one camera is selected as a reference position ant position of other cameras 10a, 10b, 10c are changed at each iteration to minimize the pre-defined cost function for joint positioning. Distance between joints can be estimated using one of available and known distance metrics. According to the selected distance metric, the cost function is prepared. Distance minimization can be performed using one of available and known optimization algorithms. When the distance between selected group of joints is minimized, the camera positions are treated as calibrated. The positions of the remaining skeleton joints (usually those, which had the highest dynamical changes of the coordinates in a sequence of video frames) are recalculated by selecting most expected position in each time stamp (related to video frame). The coordinate of the joint is selected by applying a weighted sum of joint coordinates predicted in different camera frames, selecting the highest weight for the coordinate, that was predicted from the video frame of the camera, situated at higher angle to the joint motion plane (the highest angle is received when the motion plane is parallel to the camera plane).

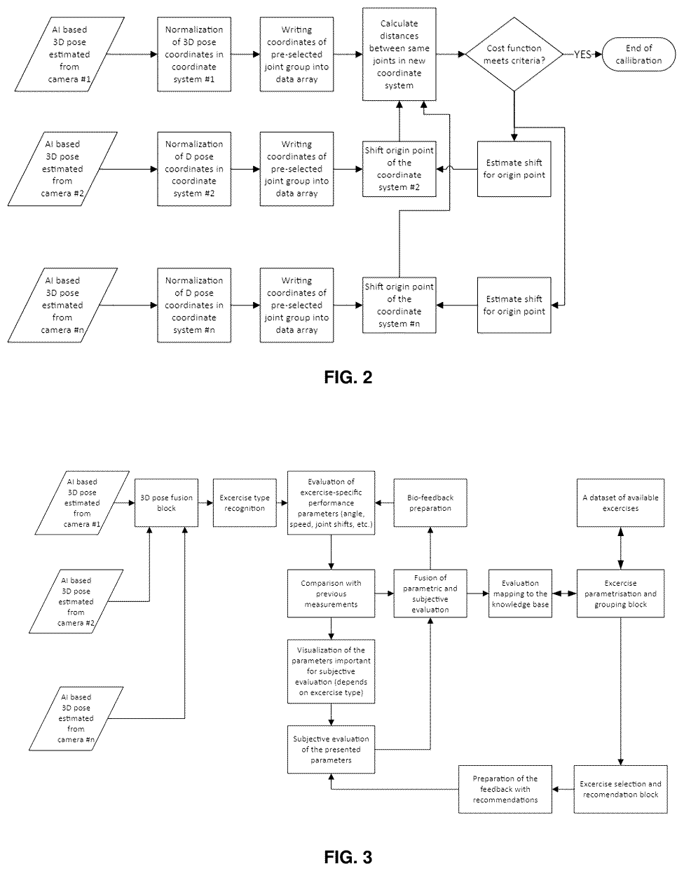

In flowchart FIG. 2 AI based 3D pose estimation results showing how 3D joint coordinates, predicted, and saved in individual camera coordinate systems are fused into single common coordinate system to perform autocalibration of the randomly placed camera views. The whole view of scope of the invention is shown in the flowchart FIG. 3.

The final human skeleton with fused and corrected positions of all 17 joints are presented to human motion analysis and parameter extraction block. Human motion analysis block calculates parameters, that are indicated by a medical professional as important to monitor during a particular exercise. Such parameters can be a speed of upper limb motion, maximum angle, displacement of joints that represent shoulder positions, etc. Therefore, the system should recognize the type of the exercise, that the patient performs.

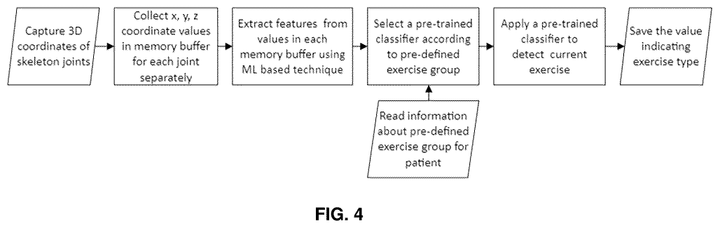

In specified flowchart FIG. 4 is shown a schematic representation of exercise type recognition. The exercise type detection is performed using one of available machine learning based classifiers. The input features, presented to the classifier, are estimated using numerical indication which skeleton joints were moving, estimated angle changes between joints during a single exercise phase, motion plane angle in respect to the human body (e.g., chest) plane, motion magnitude and other complementary features. The exercise type detection is performed from a group of exercises that were recommended for the patient by a medical professional. The list of recommended exercises is stored in a database as a list, related with a patient identification code.

After the exercise type is detected, the system selects a set of parameters, that should be monitored during patient movements. This list of parameters is pre-defined for each exercise by a medical professional in advance. Interpretation of the parameter values is performed by a subjective evaluation by medical professional. To simulate subjective evaluations of the medical professional in offline mode, a special process, presented in this invention is applied. The medical professional records subjective evaluations by observing exercises performed by different patients. A fuzzy logic-based system is applied to map subjective evaluations with skeleton joint motion related parameters, that could be measured and calculated from tracked skeleton joint positions. Membership function parameters are estimated automatically by fitting membership function to historical measurement data and to historical subjective evaluation data. Decision rules, that connects input and outputs are pre-defined once, individually for each exercise type. Subjective evaluations of the medical professional also include interpretation of biomedical feedback, explaining the processes, that are activated during the exercise and, if provided, the effect to physical and mental health.

The feedback to the user 11 is provided by visual representation of the simulated medical professional evaluations and by comparing currently measured joint motion, angle changes, and other parameters with historical measures, related with a patient. The graphical user interface visually indicated where the parameters are calculated (monitored joints are indicated, on a human manikin 3D model measured angles are indicated, the human manikin 3D model is synchronized with patient joint coordinates follows the patient movements), medical professional evaluations can be resented in form of graphs (e.g. showing the actual curve, past curves, the desired curve), color bars (e.g. using range from red to green) to indicate the progress and distance to desired values, numbers or binary indicators.

As mentioned above, since physiotherapy and rehabilitation have a dedicated purpose of improving the patient health, there is also a great significance of monitoring malfunctions in the process, by way of providing feedback to the patient of exercises performed wrongly, providing advice of corrective actions, etc.

The foregoing example of the related art and limitations related therewith is intended to be illustrative and not exclusive. Other limitations of the related art will become apparent to those of skill in the art upon a reading of the specification and a study of the figures.

1. A system for capturing, analyzing, and presenting users (11) exercise data comprising:
video cameras (10a, 10b, 10c) surrounding the user (11), capturing the user's (11) movements and processing and predicting 3D coordinates of connected human skeleton joints;
a shared hub (12) with a camera automatic calibration algorithm to map multiple human skeletons from different origin points into a single common world coordinate system, processing for identifying and correcting joint position prediction failures from a sequence of skeleton joint positions detected in consecutive video frames and from vector of features from the changes in coordinate values for each joint;
a machine learning classifier to identify the type of exercise;
a fuzzy logic-based system to map subjective evaluations to the parameters measured;
a graphical user interface displaying the monitored joints on a 3D mannequin model;
medical professional evaluations, presented in the form of graphs, color bars, numbers, and binary indicators.

2. The system of claim 1, wherein the hub comprises a synchronization system.

3. The system of claim 1, wherein the camera automatic calibration algorithm uses a distance metric to estimate the distance between joints.

4. The system of claim 2, wherein the camera automatic calibration algorithm uses human skeleton joints of the same type to minimize a pre-defined cost function for joint positioning.

5. The system of claim 4, wherein the camera automatic calibration algorithm further comprises an iterative process and a cost function.

6. The system of claim 1, wherein the graphical user interface displays a range indicate the progress and distance to desired values.

7. A method for calibrating and monitoring the execution of an exercise, comprising:
providing a plurality of video cameras around a user (11) performing an exercise;
capturing the user's (11) movements with the video cameras;
using an image processing process to predict the 3D coordinates of connected human skeleton joints;
sending the predicted joint coordinates, along with additional camera synchronization information, to a shared hub (12);
using a camera automatic calibration algorithm to map human skeletons from different origin points into a single common world coordinate system;
identifying and correcting joint position prediction failures;
taking a sequence of skeleton joint positions detected in consecutive video frames and computing a vector of features from the changes in coordinate values for each joint;
analyzing the features of the joints from all cameras to identify the joints with the lowest levels of dynamical changes in coordinates;
selecting joints of the same type (position in the skeleton) for camera automatic calibration;
estimating the camera position in the world coordinate system;
using a cost function to minimize the distance between the joint group;
assigning positions of the remaining joints using the camera frames that best capture the motions of those joints;
providing the human motion analysis and parameter extraction block with all indicated human skeleton joints in the right, fused positions;
calculating parameters that a medical specialist has identified and must be followed during the exercise;
using a machine learning classifier to identify the type of exercise the patient is working on;
using numerical data such as movement of the skeleton joints, changes in the angle between joints during a single exercise phase, motion plane angle relative to the human body plane, motion magnitude and other associated features as input features to the classifier;
selecting a set of parameters to be monitored during the exercise;
using a fuzzy logic-based system to map subjective evaluations to the parameters that have been measured;
displaying visual simulations of medical professional evaluations and comparing current joint motion, angle changes and other parameters of past values related to the patient; and
displaying the monitored joints on a 3D mannequin model that follows the patient's movements, as well as measured angles.

9. The method of claim 8, wherein the step of selecting a set of parameters to be monitored during the exercise is pre-determined by a medical professional before the exercise.

10. The method of claim 9, wherein the decision rules connecting the inputs to the outputs is pre-defined for every exercise type.

11. The method of claim 8, wherein the step of using a machine learning classifier to identify the type of exercise the patient is working on is conducted on a list of exercises recommended by a medical professional for a specific patient.

12. The method of claim 8, wherein the step of using a fuzzy logic-based system to map subjective evaluations to the parameters that have been measured includes calculating the membership function parameters automatically by fitting the data to historical measurements and subjective evaluations.

13. The method of claim 8, wherein the step of displaying visual simulations of medical professional evaluations includes presenting the evaluations.
